(19) United States
(12) Patent Application Publication
(10) Pub. No.: US 2023/0183789 A1
(43) Pub. Date: Jun. 15, 2023

(54) A METHOD OF DETECTING STRUCTURAL REARRANGEMENTS IN A GENOME

(71) Applicant: Roche Sequencing Solutions, Inc., Pleasanton, CA (US)

(72) Inventors: Daniel Klass, Livermore, CA (US); Alexander Lovejoy, Newark, CA (US)

(21) Appl. No.: 17/995,323

(22) PCT Filed: Apr. 1, 2021
(86) PCT No.: PCT/EP2021/058557

§ 371 Date: Oct. 3, 2022

Related U.S. Application Data
(60) Provisional application No. 63/005,031, filed on Apr. 3, 2020.

(51) Int. Cl. C12Q 1/6844; C12Q 1/6874; C12Q 1/6883

(52) U.S. Cl. CPC C12Q 1/6844; C12Q 1/6874; C12Q 1/6883; C12Q 2600/16; C12Q 2600/156

(57) ABSTRACT

Disclosed are methods and compositions for detecting structural rearrangements in a genome using rearrangement-specific enrichment probes or rearrangement- specific amplification primers.

### FIELD OF THE INVENTION

The invention relates to the field of nucleic acid sequencing. More specifically, the invention relates to the field of detecting genomic rearrangements by sequencing.

### BACKGROUND OF THE INVENTION

A significant percentage of cancer genomes have structural aberrations, either a copy number amplification (CNA, where large portions of the genome are tandemly repeated), copy number deletions (CND, where large portions of the genome are removed), translocations (fusions with other portions of the genome) tandem repeats (in which regions of the genome smaller than a gene are tandemly replicated) or deletions (in which regions smaller than a gene are deleted). The ability to detect these variants can be helpful in detecting and diagnosing cancer, in tracking tumor burden over time, and for identifying the best individualized treatment for a cancer patients.

Existing method of detecting genomic rearrangements involve cumbersome multi-step procedures such as haplotype fusion PCR and ligation haplotyping, see Turner et al., (2008) Long range, high throughput haplotype determination via haplotype fusion PCR and ligation haplotyping, Nucl. Acids Res. 36:e82.

Current sequencing-based techniques for identification of these structural aberrations exist but often require large amounts of sequencing. Since the cost of next-generation sequencing is typically the primary driver of assay cost, the ability to identify such structural aberrations with less sequencing would greatly reduce cost of assays and increase patient access to these diagnostic tools.

### SUMMARY OF THE INVENTION

The invention is a method of detecting a rare genomic rearrangement such as a fusion, deletion or copy number amplification in a sample using specially arranged pairs of forward and reverse primers.

In one embodiment, the invention is a method of detecting a genomic rearrangement in a sample, the method comprising contacting a sample containing nucleic acids from a genome with one or more pairs of a forward and a reverse oligonucleotide primers wherein the binding sites for the primers in a reference genome are not adjacent or not inward-facing, and wherein the position of the binding sites for the primers in a genome comprising a genomic rearrangement is adjacent and inward-facing to allow exponentially amplifying the nucleic acid comprising the rearrangement with the forward and reverse primers, and exponentially amplifying the nucleic acid comprising the rearrangement thereby detecting the rearrangement. The method may further comprise a step of sequencing the amplified nucleic acids thereby detecting the rearrangement. Adjacent may mean less than 2000 base pairs apart in cellular genomic DNA or less than 175 base pairs apart in cell-free DNA.

In some embodiments, the genomic rearrangement is a gene fusion and the binding sites for the forward and reverse primers are located on different chromosomes in a reference genome but are located on the same chromosome in the genome comprising the gene fusion. In some embodiments, the genomic rearrangement is a deletion and the binding sites for the forward and reverse primers are not adjacent in a reference genome but are adjacent in a genome comprising the deletion. In some embodiments, the genomic rearrangement creates a breakpoint sequence and one of the binding sites for the forward and reverse primers spans the breakpoint sequence. In some embodiments, the genomic rearrangement is an amplification and at least one of the copies of the forward primer-binding site and one of the copies of the reverse primer-binding site are inward-facing in the genome comprising the amplification.

In some embodiments, the invention is a method of simultaneously interrogating a sample for one or more types of genomic rearrangements, the method comprising: contacting a sample containing nucleic acids from a genome with one or more pairs of a forward and a reverse oligonucleotide primers wherein the binding sites for the primers in a reference genome are not adjacent or not inward-facing, and wherein the position of the binding sites for the primers in a genome comprising a genomic rearrangement is adjacent and inward-facing to allow exponentially amplifying the nucleic acid comprising the rearrangement with the forward and reverse primers; exponentially amplifying the nucleic acid comprising the rearrangement; forming a library of amplified nucleic acids; sequencing the nucleic acids in the library thereby detecting one or more genomic rearrangements in the sample. In some embodiments, the method further comprises aligning the sequencing reads with the reference genome to determine the genomic source of the genomic rearrangement.

In some embodiments, one or more pairs of a forward and a reverse oligonucleotide primers comprise: for at least one pair of forward and reverse primers, the binding sites for the forward and reverse primers are located on different chromosomes in a reference genome but are located on the same chromosome in the genome comprising a gene fusion; and for at least one pair of forward and reverse primers, one of the binding sites for the forward and reverse primers spans a breakpoint sequence of a genomic rearrangement; and for at least one pair of forward and reverse primers, one of the copies of the forward primer binding site and one of the copies of the reverse primer binding site are inward-facing in the genome comprising gene amplification.

In some embodiments, the rearrangements include fusions involving one or more genes selected from ALK, PPARG, BRAF, EGFR, FGFR1, FGFR2, FGFR3, MET, NRG1, NTRK1, NTRK2, NTRK3, RET, ROS1, AXL, PDGFRA, PDGFB, ABL1, ABL2, AKT1, AKT2, AKT3, ARHGAP26, BRD3, BRD4, CRLF2, CSF1R, EPOR, ERBB2, ERBB4, ERG, ESR1, ESRRA, ETV1, ETV4, ETV5, ETV6, EWSR1, FGR, IL2RB, INSR, JAK1, JAK2, JAK3, KIT, MAML2, MAST1, MAST2, MSMB, MUSK, MYB, MYC, NOTCH1, NOTCH2, NUMBL, NUT, PDGFRB, PIK3CA, PKN1, PRKCA, PRKCB, PTK2B, RAF1, RARA, RELA, RSPO2, RSPO3, SYK, TERT, TFE3, TFEB, THADA, TMPRSS2, TSLP, TY, BCL2, BCL6, BCR, CAMTA1, CBFB, CCNB3, CCND1, CIC, CRFL2, DUSP22, EPC1, FOXO1, FUS, GLI1, GLIS2, HMGA2, JAZF1, KMT2A, MALT1, MEAF6, MECOM, MKL1, MKL2, MTB, NCOA2, NUP214, NUP98, PAX5, PDGFB, PICALM, PLAG1, RBM15, RUNX1, RUNX1T1, SS18, STAT6, TAF15, TAL1, TCF12, TCF3, TFG, TYK2, USP6, YWHAE, AR, BRCA1, BRCA2, CDKN2A, ERB84, FLT3, KRAS, MDM4, MYBL1, NF1, NOTCH4, NUTM1, PRKACA, PRKACB, PTEN, RAD51B, and RB1, and deletions or duplications involving one or more genes selected from EGFR, ERBB2, MET, MYC, BCL2, and BCL6. In some embodiments, the method further comprises contacting the sample with one or more pairs of control forward and a reverse oligonucleotide primers wherein the binding sites for the primers in a reference genome are adjacent and not inward-facing to allow exponentially amplifying the non-rearranged reference sequence.

In some embodiments, forming a library comprises: attaching adaptors comprising barcodes, and sequencing comprises determining the sequence of tagged library nucleic acids, grouping the sequence by tags into families, determining consensus read for each family, aligning the consensus read to the reference genome thereby detecting a genomic rearrangement.

In some embodiments, the invention is a method of detecting a genomic rearrangement in a sample, the method comprising: forming a library of nucleic acids comprising at least one adaptor; hybridizing to a library nucleic acid a first primer of a primer pair, wherein the first primer hybridizes on one side of a genomic rearrangement and also comprises a capture moiety; extending the hybridized first primer, thereby producing a first primer extension complex comprising the sequence of the genomic rearrangement and further comprising a capture moiety, capturing the first primer extension product via the capture moiety; hybridizing to the captured nucleic acid a second primer of a primer pair wherein second primer hybridizes to the opposite strand on the opposite side of the genomic rearrangement relative to the first primer and adjacent to the first primer in the rearranged genome but not in the reference genome; forming a copy of the captured rearranged nucleic acid; sequencing the copy of the rearranged nucleic acid thereby detecting the genomic rearrangement.

In some embodiments, the invention is a method of enriching for a sequence containing a genomic rearrangement in a sample, the method comprising: hybridizing to nucleic acids in a sample a first primer, wherein the first primer hybridizes on one side of a genomic rearrangement and also comprises a capture moiety; extending the hybridized first primer, thereby producing a first primer extension complex comprising the sequence of the genomic rearrangement and further comprising the capture moiety; capturing the first primer extension product via the capture moiety; hybridizing to the captured nucleic acid a second primer, wherein second primer hybridizes to the same strand on the same side of the genomic rearrangement relative to the first primer in the rearranged genome but not in the reference genome, and also comprises a barcode; extending the hybridized second primer, thereby producing a second primer extension complex and displacing the first primer extension complex comprising the capture moiety; hybridizing to the second primer extension complex a third primer wherein the third primer hybridizes to the opposite strand on the opposite side of the genomic rearrangement relative to the second primer and adjacent to the second primer in the rearranged genome but not in the reference genome; extending the third primer thereby forming a double-stranded product comprising the sequence of a rearrangement thereby enriching for the genomic rearrangement. The capture moiety of the first oligonucleotide may be a capture sequence, a chemical moiety for which a ligand is available or an antigen for which an antibody is available. The capture moiety is a capture sequence complementary to a capture oligonucleotide, which comprises a modified nucleotide increasing the melting temperature of the capture oligonucleotide, for example, 5-methyl cytosine, 2,6-diaminopurine, 5-hydroxybutynl-2′-deoxyuridine, 8-aza-7-deazaguanosine, a ribonucleotide, a 2′O-methyl ribonucleotide and locked nucleic acid. In some embodiments, the first oligonucleotide is bound to a solid support via the capture moiety prior to hybridizing the first oligonucleotide to the target nucleic acid. In some embodiments, the method also includes sequencing the double-stranded product thereby detecting the genomic rearrangement. The sequencing may comprise determining the sequence of double-stranded nucleic acids and attached barcodes, grouping the sequence by barcodes into families, determining consensus read for each family, aligning the consensus read to the reference genome thereby detecting a genomic rearrangement.

In some embodiments, the invention is a method of detecting a structural variation in RNA transcripts in a sample, comprising: obtaining nucleic acids from a sample; reverse transcribing RNA transcripts into cDNA strands with a first primer positioned adjacent to a site of a genomic rearrangement; hybridizing to the cDNA strands a second primer wherein the second primer hybridizes to the opposite strand on the opposite side of the genomic rearrangement relative to the first primer and adjacent to the first primer in a rearranged genome but not in a reference genome to enable exponential amplification of a rearranged genome sequence but not of a reference genome sequence; and amplifying the cDNA to produce amplicons thereby detecting genomic rearrangement in the RNA transcripts.

In some embodiments, the invention is a method for detecting a genomic rearrangement in a nucleic acid in a sample, comprising: partitioning a sample comprising nucleic acids from a genome into a plurality of reaction volumes; wherein each reaction volume comprises (i) a first primer that is capable of hybridizing on one side of a genomic rearrangement, (ii) a second primer that is capable of hybridizing to the opposite strand on the opposite side of the genomic rearrangement relative to the first primer and adjacent to the first primer in the rearranged genome but not in a reference genome, and (iii) a detectably-labeled first probe capable of hybridizing to an amplicon of the first and second primers; performing an amplification reaction with the first and the second primers, wherein the reaction comprises a step of detection with the probe; determining a number of reaction volumes where the first probe has been detected thereby detecting the genomic rearrangement. The reaction volumes may be droplets. In some embodiments, the reaction volumes further comprise a third primer that is capable of hybridizing to the opposite strand relative to the first primer and adjacent to the first primer in the reference genome but not in the rearranged genome, and a second detectably labeled probe capable of hybridizing to an amplicon of the first and third primers but not the amplicon of the first and second primers, and the method further comprising determining a ratio of reaction volumes where the first probe has been detected to the number of reaction volumes where the second probe has been detected thereby detecting the frequency of genomic rearrangement. In some embodiments, the first probe hybridizes to a sequence in a rearranged genome but not a reference genome. In some embodiments, the second probe hybridizes to a sequence in a reference genome but not in a rearranged genome. The first and second probes may have different detectable labels. A label can be for example, a combination of a fluorophore and a quencher.

### BRIEF DESCRIPTION OF THE DRAWINGS

FIG. 1 is a diagram of primers flanking a genomic rearrangement.

FIG. 2 is a diagram of primers designed to detect a fusion event.

FIG. 3 is a diagram of primers designed to detect a deletion event.

FIG. 4 is a diagram of primers designed to detect an amplification event.

FIG. 5 is a diagram of detection a rearrangement by Primer Extension Target Enrichment (PETE).

### DETAILED DESCRIPTION OF THE INVENTION

### Definitions

Unless defined otherwise, technical and scientific terms used herein have the same meaning as commonly understood by a person of ordinary skill in the art. See, Sambrook et al., Molecular Cloning, A Laboratory Manual, 4^{th} Ed. Cold Spring Harbor Lab Press (2012).

The following definitions are provided to facilitate understanding of the present disclosure.

The term “adaptor” refers to a nucleotide sequence that may be added to another sequence in order to import additional elements and properties to that sequence. The additional elements include without limitation: barcodes, primer binding sites, capture moieties, labels, secondary structures.

The term “barcode” refers to a nucleic acid sequence that can be detected and identified. Barcodes can generally be 2 or more and up to about 50 nucleotides long. Barcodes are designed to have at least a minimum number of differences from other barcodes in a population. Barcodes can be unique to each molecule in a sample or unique to the sample and be shared by multiple molecules in the sample. The term “multiplex identifier,” “MID” or “sample barcode” refer to a barcode that identifies a sample or a source of the sample. As such, all or substantially all, MID barcoded polynucleotides from a single source or sample will share an MID of the same sequence; while all, or substantially all (e.g., at least 90% or 99%), MID barcoded polynucleotides from different sources or samples will have a different MID barcode sequence. Polynucleotides from different sources having different MIDs can be mixed and sequenced in parallel while maintaining the sample information encoded in the MID barcode. The term “unique molecular identifier” or “UID,” refer to a barcode that identifies a polynucleotide to which it is attached. Typically, all, or substantially all (e.g., at least 90% or 99%), UID barcodes in a mixture of UID barcoded polynucleotides are unique.

The term “DNA polymerase” refers to an enzyme that performs template-directed synthesis of polynucleotides from deoxyribonucleotides. DNA polymerases include prokaryotic Pol I, Pol II, Pol III, Pol IV and Pol V, eukaryotic DNA polymerase, archaeal DNA polymerase, telomerase and reverse transcriptase. The term “thermostable polymerase,” refers to an enzyme that is useful in exponential amplification of nucleic acids by polymerase chain reaction (PCR) by virtue of the enzyme being heat resistant. A thermostable enzyme retains sufficient activity to effect subsequent polynucleotide extension reactions and does not become irreversibly denatured (inactivated) when subjected to the elevated temperatures for the time necessary to effect denaturation of double-stranded nucleic acids. In some embodiments, the thermostable polymerases from species Thermococcus, Pyrococcus, Sulfolobus Methanococcus and other archaeal B polymerases. In some cases, the nucleic acid (e.g., DNA or RNA) polymerase may be a modified naturally occurring Type A polymerase. A further embodiment of the invention generally relates to a method wherein a modified Type A polymerase, e.g., in a primer extension, end-modification (e.g., terminal transferase, degradation, or polishing), or amplification reaction, may be selected from any species of the genus Meiothermus, Thermotoga, or Thermomicrobium. Another embodiment of the invention generally pertains to a method wherein the polymerase, e.g., in a primer extension, end-modification (e.g., terminal transferase, degradation or polishing), or amplification reaction, may be isolated from any of Thermus aquaticus (Taq), Thermus thermophilus, Thermus caldophilus, or Thermus filiformis. A further embodiment of the invention generally encompasses a method wherein the modified Type A polymerase, e.g., in a primer extension, end-modification (e.g., terminal transferase, degradation, or polishing), or amplification reaction, may be isolated from Bacillus stearothermophilus, Sphaerobacter thermophilus, Dictoglomus thermophilum, or Escherichia coli. In another embodiment, the invention generally relates to a method wherein the modified Type A polymerase, e.g., in a primer extension, end-modification (e.g., terminal transferase, degradation, or polishing), or amplification reaction, may be a mutant Taq-E507K polymerase. Another embodiment of the invention generally pertains to a method wherein a thermostable polymerase may be used to effect amplification of the target nucleic acid.

The term “enrichment” refers to increasing the relative amount of target molecules in the plurality of molecules. Enrichment may increase the relative amount of target molecules up to total or near total exclusion of non-target molecules. Examples of enrichment of target nucleic acids include linear hybridization capture, amplification, exponential amplification (PCR) and Primer Extension Target Enrichment (PETE), see e.g., U.S. Application Ser. Nos. 14/910,237, 15/228,806, 15/648,146 and International Application Ser. No. PCT/EP2018/085727.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologues, SNPs, and complementary sequences as well as the sequence explicitly indicated.

The term “primer” refers to an oligonucleotide, which binds to a specific region of a single-stranded template nucleic acid molecule and initiates nucleic acid synthesis via a polymerase-mediated enzymatic reaction. Typically, a primer comprises fewer than about 100 nucleotides and preferably comprises fewer than about 30 nucleotides. A target-specific primer specifically hybridizes to a target polynucleotide under hybridization conditions. Such hybridization conditions can include, but are not limited to, hybridization in isothermal amplification buffer (20 mM Tris-HCl, 10 mM (NH₄)₂SO₄), 50 mM KCl, 2 mM MgSO₄, 0.1% TWEEN® 20, pH 8.8 at 25° C.) at a temperature of about 40° C. to about 70° C. In addition to the target-binding region, a primer may have additional regions, typically at the 5′-poriton. The additional region may include universal primer binding site or a barcode. For exponential amplification to take place, the primers must be inward-facing, i.e., hybridizing to opposite strands of the target nucleic acid with 3′-ends facing towards each other. This orientation of amplification primers is sometimes referred to as “correct orientation.” Further, for exponential amplification to take place, the primers hybridize to the target nucleic acid within a suitable distance from each other. Under standard PCR conditions, primers hybridizing to opposite strands farther than 2000 base pairs apart would not yield a sufficient amount of product. In the case of a cfDNA sample, the typical fragment size 175 base pairs apart, therefore primers hybridizing to opposite strands farther than 175 base pairs apart would typically not yield amplified product.

The term “reference genome” and “reference genome sequence” refer to entire human genome sequence (“genome build”) released to the public and periodically updated by the National Center for Biotechnology Information (NCBI), currently build GRCh38. The reference genome is searchable by chromosome location and sequence to enable comparing a sequence from an individual sample and identifying any sequence changes in the sample.

The terms “rearranged genome” refers to a genome comprising one or more rearrangements when compared to a reference genome. It is understood that a rearranged genome also contains non-rearranged sequences at other loci not involved in rearrangements. Such loci in the rearranged genome have the same sequence as the corresponding reference genome loci. The term “rearranged genome sequence” refers to the rearranged sequence in the rearranged genome.

The term “genomic rearrangement” refers to a change in the genome sequence as compared to the reference genome. Rearrangement is a change involving more than a few nucleotides. Examples of genomic rearrangement include copy number amplification (CNA, where large portions of the genome are tandemly repeated), copy number deletions (CND, where large portions of the genome are removed), translocations (fusions with other portions of the genome) tandem repeats (in which regions of the genome smaller than a gene are tandemly replicated) or deletions (in which regions smaller than a gene are deleted). In contract, a single nucleotide variation (SNV) is not a genomic rearrangement.

The term “sample” refers to any biological sample that comprises nucleic acid molecules, typically comprising DNA or RNA. Samples may be tissues, cells or extracts thereof, or may be purified samples of nucleic acid molecules. The term “sample” refers to any composition containing or presumed to contain target nucleic acid. Use of the term “sample” does not necessarily imply the presence of target sequence among nucleic acid molecules present in the sample. The sample can be a specimen of tissue or fluid isolated from an individual for example, skin, plasma, serum, spinal fluid, lymph fluid, synovial fluid, urine, tears, blood cells, organs and tumors, and also to samples of in vitro cultures established from cells taken from an individual, including the formalin-fixed paraffin embedded tissues (FFPET) and nucleic acids isolated therefrom. A sample may also include cell-free material, such as cell-free blood fraction that contains cell-free DNA (cfDNA) or circulating tumor DNA (ctDNA). The sample can be collected from a non-human subject or from the environment.

The term “target” or “target nucleic acid” refer to the nucleic acid of interest in the sample. The sample may contain multiple targets as well as multiple copies of each target.

The term “universal primer” refers to a primer that can hybridize to a universal primer binding site. Universal primer binding sites can be natural or artificial sequences typically added to a target sequence in a non-target-specific manner.

The invention is method of detecting genomic rearrangements also known as structural aberrations in a genome utilizing an amplicon-based approach. The method allows detecting genomic rearrangements with minimal sequencing depth. Any time a structural aberration such as a genomic rearrangement occurs, at least one breakpoint is present in the rearranged genome. A breakpoint is a point at which genomic regions that are normally not adjacent become adjacent. The instant invention is a method of detecting genomic rearrangements that enables to amplify and detect such breakpoints related to genomic rearrangements. The method of the invention is designed to work with any two-primer amplification approach utilizing at least one forward primer and at least one reverse primer. Examples of such approaches include Polymerase Chain Reaction (PCR) and Primer Extension Target Enrichment (PETE).

The forward and reverse primers are designed around potential regions of copy number amplifications, copy number deletions, fusions, tandem repeats or large deletions. In the absence of a genomic rearrangement, the forward and reverse primers are not adjacent or incorrectly oriented relative to each other and are not capable of supporting amplification so that no amplicon is made. In the presence of a genomic rearrangement, the forward and reserve primer enable the formation of an amplicon that can be detected thereby detecting the rearrangement.

The present invention utilizes a sample containing nucleic acids. In some embodiments, the sample is derived from a subject or a patient. In some embodiments the sample may comprise a fragment of a solid tissue or a solid tumor derived from the subject or the patient, e.g., by biopsy. The sample may also comprise body fluids (e.g., urine, sputum, serum, plasma or lymph, saliva, sputum, sweat, tear, cerebrospinal fluid, amniotic fluid, synovial fluid, pericardial fluid, peritoneal fluid, pleural fluid, cystic fluid, bile, gastric fluid, intestinal fluid, or fecal samples). The sample may comprise whole blood or blood fractions where normal or tumor cells may be present. In some embodiments, the sample, especially a liquid sample may comprise cell-free material such as cell-free DNA or RNA including cell-free tumor DNA or tumor RNA of cell-free fetal DNA or fetal RNA. In some embodiments, the sample is a cell-free sample, e.g., cell-free blood-derived sample where cell-free tumor DNA or tumor RNA or cell-free fetal DNA or fetal RNA are present. In other embodiments, the sample is a cultured sample, e.g., a culture or culture supernatant containing or suspected to contain nucleic acids derived from the cells in the culture or from an infectious agent present in the culture. In some embodiments, the infectious agent is a bacterium, a protozoan, a fungus, a virus or a mycoplasma.

Target nucleic acids are the nucleic acid of interest that may be present in the sample. Each target is characterized by its nucleic acid sequence. The present invention enables detection of one or more RNA or DNA targets. In some embodiments, the DNA target nucleic acid is a gene or a gene fragment (including exons and introns) or an intergenic region, and the RNA target nucleic acid is a transcript or a portion of the transcript to which target-specific primers hybridize. In some embodiments, the target nucleic acid contains a locus of a genetic variant, e.g., a polymorphism, including a single nucleotide polymorphism or variant (SNP of SNV), or a genetic rearrangement resulting e.g., in a gene fusion. In some embodiments, the target nucleic acid comprises a biomarker, i.e., a gene whose variants are associated with a disease or condition. For example, the target nucleic acids can be selected from panels of disease-relevant markers described in U.S. Pat. Application Ser. No. 14/774,518 filed on Sep. 10, 2015. Such panels are available as AVENIO ctDNA Analysis kits (Roche Sequencing Solutions, Pleasanton, Cal.) Of special interest are the genes known to undergo rearrangements in tumors. For example, ALK, RET, ROS, FGFR2, FGFR3 and NTRK1 are known to undergo fusions resulting in an abnormally active kinase phenotype. EGFR, ERBB2, MET, MYC, BCL2, and BCL6 are among genes known to be involved in rearrangements involving a change in copy number. (Li et al. Nature 2020, Hieronymus et al. eLife 2017). Genes known or expected to undergo fusions relevant for cancer include ALK, PPARG, BRAF, EGFR, FGFR1, FGFR2, FGFR3, MET, NRG1, NTRK1, NTRK2, NTRK3, RET, ROS1, AXL, PDGFRA, PDGFB , ABL1, ABL2, AKT1, AKT2, AKT3, ARHGAP26, BRD3, BRD4, CRLF2, CSF1R, EPOR, ERBB2, ERBB4, ERG, ESR1, ESRRA, ETV1, ETV4, ETV5, ETV6, EWSR1, FGR, IL2RB, INSR, JAK1, JAK2, JAK3, KIT, MAML2, MAST1, MAST2, MSMB, MUSK, MYB, MYC, NOTCH1, NOTCH2, NUMBL, NUT, PDGFRB, PIK3CA, PKN1, PRKCA, PRKCB, PTK2B, RAF1, RARA, RELA, RSPO2, RSPO3, SYK, TERT, TFE3, TFEB, THADA, TMPRSS2, TSLP, TY, BCL2, BCL6, BCR, CAMTA1, CBFB, CCNB3, CCND1, CIC, CRFL2, DUSP22, EPC1, FOXO1, FUS, GLI1, GLIS2, HMGA2, JAZF1, KMT2A, MALT1, MEAF6, MECOM, MKL1, MKL2, MTB, NCOA2, NUP214, NUP98, PAX5, PDGFB, PICALM, PLAG1, RBM15, RUNX1, RUNX1T1, SS18, STAT6, TAF15, TAL1, TCF12, TCF3, TFG, TYK2, USP6, YWHAE, AR, BRCA1, BRCA2, CDKN2A, ERB84, FLT3, KRAS, MDM4, MYBL1, NF1, NOTCH4, NUTM1, PRKACA, PRKACB, PTEN, RAD51B, and RB1.

In some embodiments, the target nucleic acid is RNA (including mRNA, microRNA, viral RNA). In such embodiments, as further discussed below a reverse transcription step is employed. In other embodiments, the target nucleic acid is DNA, including cellular DNA or cell-free DNA (cfDNA) including circulating tumor DNA (ctDNA) and cell-free fetal DNA. The target nucleic acid may be present in a short or long form. In some embodiments, longer target nucleic acids are fragmented by enzymatic or physical treatment as described below. In some embodiments, the target nucleic acid is naturally fragmented, e.g., includes circulating cell-free DNA (cfDNA) or chemically degraded DNA such as the one found in chemically preserved or ancient samples.

In some embodiments, the invention comprises a step of nucleic acid isolation. Generally, any method of nucleic acid extraction that yields isolated nucleic acids comprising DNA or RNA may be used as both long and short nucleic acid starting material is suitable for use in the method of the invention. Genomic DNA or RNA may be extracted from tissues, cells, liquid biopsy samples (including blood or plasma samples) using solution-based or solid-phase based nucleic acid extraction techniques. Nucleic acid extraction can include detergent-based cell lysis, denaturation of nucleoproteins, and optionally removal of contaminants. Extraction of nucleic acids from preserved samples may further include a step of deparaffinization. Solution based nucleic acid extraction methods may comprise salting out methods or organic solvent or chaotrope methods. Solid-phase nucleic extraction methods can include but are not limited to silica resin methods, anion exchange methods or magnetic glass particles and paramagnetic beads (KAPA Pure Beads, Roche Sequencing Solutions, Pleasanton, Cal.) or AMPure beads (Beckman Coulter, Brea, Cal.)

A typical extraction method involves lysis of tissue material and cells present in the sample. Nucleic acids released from the lysed cells can be bound to a solid support (beads or particles) present in solution or in a column, or membrane where the nucleic acids may undergo one or more washing steps to remove contaminants including proteins, lipids and fragments thereof from the sample. Finally, the bound nucleic acids can be released from the solid support, column or membrane and stored in an appropriate buffer until ready for further processing. Because both DNA and RNA must be isolated, no nucleases may be used and care should be taken to inhibit any nuclease activity during the purification process.

In some embodiments, nucleic acid isolation utilizes epitachophoresis (ETP) as described in PCT/EP2019/077714 filed on Oct. 14, 2019 and PCT/EP2018/081049 filed on Nov. 13, 2018. ETP utilizes a device with a circular arrangement of electrodes where the nucleic acid migrates and concentrates between a leading electrolyte and a trailing electrolyte. The circular configuration allows concentrating nucleic acids in a very small volume collected in the center of the device. The use of ETP is especially advantageous for blood plasma samples containing small amounts of cell-free nucleic acid in a large volume.

In some embodiments, the input DNA or input RNA require fragmentation. In such embodiments, RNA may be fragmented by a combination of heat and metal ions, e.g., magnesium. In some embodiments, the sample is heated to 85°-94° C. for 1-6 minutes in the presence of magnesium. (KAPA RNA HyperPrep Kit, KAPA Biosystems, Wilmington, Mass). DNA can be fragmented by physical means, e.g., sonication, using commercially available instruments (Covaris, Woburn. Mass.) or enzymatic means (KAPA Fragmentase Kit, KAPA Biosystems).

In some embodiments, the isolated nucleic acid is treated with DNA repair enzymes. In some embodiments, the DNA repair enzymes comprise a DNA polymerase which has 5′-3′ polymerase activity and 3′-5′ single stranded exonuclease activity, a polynucleotide kinase which adds a 5′ phosphate to the dsDNA molecule, and a DNA polymerase which adds a single dA base at the 3′ end of the dsDNA molecule. The end repair/A-tailing kits are available e.g., Kapa Library Preparation, kits including KAPA Hyper Prep and KAPA HyperPlus (Kapa Biosystems, Wilmington, Mass.).

In some embodiments, the DNA repair enzymes target damaged bases in the isolated nucleic acids. In some embodiments, sample nucleic acid is partially damaged DNA from preserved samples, e.g., formalin-fixed paraffin embedded (FFPET) samples. Deamination and oxidation of bases can result in an erroneous base read during the sequencing process. In some embodiments, the damaged DNA is treated with uracil N-DNA glycosylase (UNG/UDG) and/or 8-oxoguanine DNA glycosylase.

In some embodiments, the target nucleic acid is RNA, e.g., messenger RNA (mRNA) from a sample. In this embodiment, the method described in relation to DNA including double-stranded DNA from the sample is used, except the method comprises a preliminary step of reverse transcription. In some embodiments, reverse transcription is initiated by a gene-specific primer annealing to the RNA adjacent to the site of the rearrangement expected to be present in mRNA. In other embodiments, reverse transcription is initiated by a poly-T primer. In yet other embodiments, reverse transcription is initiated by a random primer, e.g., a random hexamer primer. In yet other embodiments, reverse transcription is initiated by a combination primer comprising a poly-T sequence and a random sequence.

In some embodiments, the invention comprises an amplification step. The isolated nucleic acids can be amplified prior to further processing. This step can involve linear or exponential amplification. Amplification may be isothermal or involve thermocycling. In some embodiments, the amplification is exponential and involves PCR. In some embodiments, gene-specific primers are used for amplification. In other embodiments, universal primer binding sites are added to target nucleic acid e.g., by ligating an adaptor comprising the universal primer binding sites. All adaptor-ligated nucleic acids have the same universal primer binding sites and can be amplified with the same set of primers. The number of amplification cycles where universal primers are used can be low but also can be 10, 20 or as high as about 30 or more cycles, depending on the amount of product needed for the subsequent steps. Because PCR with universal primers has reduced sequence bias, the number of amplification cycles need not be limited to avoid amplification bias.

In some embodiments, the invention involves an amplification step utilizing a forward and a reverse primer. One or both of the forward and reverse primers may be target-specific. A target specific primer comprises at least a portion that is complementary to the target nucleic acid. If additional sequences are present, such as a barcode or a second primer binding site, they are typically located in the 5′-portion of the primer. The target may be a gene sequence (coding or non-coding) or a regulatory sequence present in RNA such as an enhancer or a promoter. The target may also be an inter-genic sequence.

In some embodiments, amplification is not a rearrangement-specific step but serves to increase (amplify) the amount of the starting material or the final product of the rearrangement- specific amplification. In such embodiments, amplification primers are either target-specific but not rearrangement specific. For example, the primers are universal, e.g., can amplify all nucleic acids in the sample regardless of the target sequence as long as a universal primer binding site has been introduced into the nucleic acid. Universal primers anneal to universal primer binding sites added to the nucleic acids in the sample by extending a primer having the universal primer binding site in the 5′-region of the primer or by ligating an adaptor comprising the universal primer binding site.

In the context of the present invention, the rearrangement-specific target-specific primers are positioned near the breakpoint of a genomic rearrangement as further described below. For exponential amplification to occur, the primers must be located at a suitable distance from each other and be opposite-facing, e.g., hybridizing to opposite strands of the target nucleic acids with 3′-ends facing towards each other and capable of being extended towards to copy the sequence between the forward and reverse primer binding sites. Exponential amplification by polymerase chain reaction (PCR) is not efficient if the distance between the forward and reverse primers exceeds 2000 bases. Furthermore, exponential amplification will not be successful if the distance between primers exceeds the average size of the DNA molecule in the sample (e.g. ~175 bp is the typical size of a cfDNA molecule). In the context of the instant invention, the forward and reverse primers are designed so that efficient exponential amplification occurs only in the presence of a genomic rearrangement in the target sequence. In the absence of the predicted genomic rearrangement, the amplification does not occur or is inefficient so as to fall below the level of detection or produce a signal clearly distinguishable from that of efficient amplification.

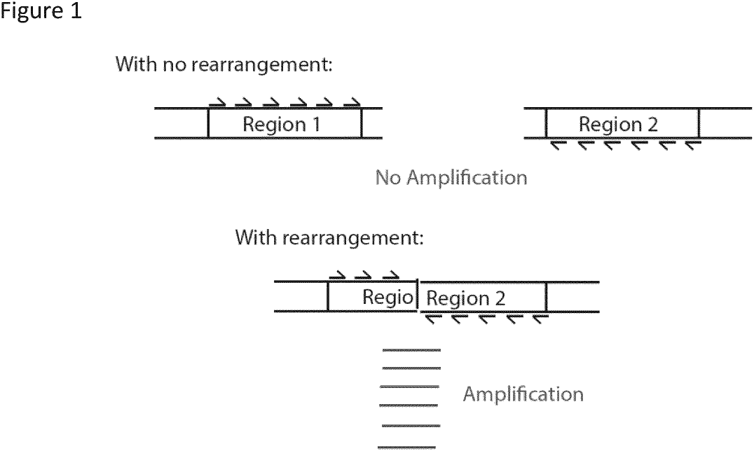

In some embodiments, the primers are tiled. Instead of just one forward primer and one reverse primer, a series of tandemly arranged forward primers, and a series of tandemly arranged reverse primers is used. In some embodiments, a single forward primer is paired with a series of tiled reverse primers. In other embodiments, a single reverse primer is paired with a series of tiled forward primers. In yet other embodiments, a series of tiled reverse primers is paired with a series of tiled forward primers. (FIGS. 1, 2 or 3). The tiled primer configuration is especially advantageous where an exact location of the breakpoint in not known. For example, some genes (ALK, ROS and NTRK1) are known to be involved in a variety fusion events, each with a different breakpoint within the gene sequence.

In some embodiments, the invention is a library of nucleic acids enriched for rearrangement-specific nucleic acids as described herein. The library comprises double-stranded nucleic acid molecules flanked by adaptor sequences described herein. The library nucleic acids may comprise elements such as barcodes and universal primer binding sites present in adaptor sequences as described herein below. In some embodiments, the additional elements are present in adaptors and are added to the library nucleic acids via adaptor ligation. In other embodiments, some or all of the additional elements are present in amplification primers and are added to the library nucleic acids prior to adaptor ligation by extension of the primers. The utility of adaptors and amplification primers for introducing additional elements in to a library of nucleic acids to be sequenced has been described e.g., in U.S. Pat. Nos. 9476095, 9260753, 8822150, 8563478, 7741463, 8182989 and 8053192.

In some embodiments, the library is formed from nucleic acids in the sample prior to the use of rearrangement-specific primers described herein. In this embodiment, adaptor molecules are added to all nucleic acids in the sample. Rearrangement-specific enrichment uses library molecules as starting material. In some embodiments, universal amplification (with universal primers hybridizing to primer binding sites located in adaptors) takes place prior to rearrangement-specific amplification or enrichment. The universal amplification increases the amount of starting material for rearrangement-specific amplification or enrichment.

In other embodiments, the library is formed from products of rearrangement-specific enrichment conducted as described herein. In variations of this embodiment, adaptor sequences are added to the products of rearrangement-specific enrichment either by ligation of adaptors or by virtue of adaptor sequences being present in the 5′-portions of rearrangement-specific primers. In some embodiments, rearrangement-specific amplification with rearrangement-specific primers is followed by universal amplification with universal primers.

In some embodiments, the invention utilizes an adaptor nucleic acid. The adaptor may be added to the nucleic acid by a blunt-end ligation or a cohesive end ligation. In some embodiments, the adaptor may be added by single-strand ligation method. In some embodiments, the adaptor molecules are in vitro synthesized artificial sequences. In other embodiments, the adaptor molecules are in vitro synthesized naturally occurring sequences. In yet other embodiments, the adaptor molecules are isolated naturally occurring molecules or isolated non-naturally occurring molecules.

In the case of adaptor added by ligation, the adaptor oligonucleotide can have overhangs or blunt ends on the terminus to be ligated to the target nucleic acid. In some embodiments, the adaptor comprises blunt ends to which a blunt-end ligation of the target nucleic acid can be applied. The target nucleic acids may be blunt-ended or may be rendered blunt-ended by enzymatic treatment (e.g., “end repair.”). In other embodiments, the blunt-ended DNA undergoes A-tailing where a single A nucleotide is added to the 3′-end of one or both blunt ends. The adaptors described herein are made to have a single T nucleotide extending from the blunt end to facilitate ligation between the nucleic acid and the adaptor. Commercially available kits for performing adaptor ligation include AVENIO ctDNA Library Prep Kit or KAPA HyperPrep and HyperPlus kits (Roche Sequencing Solutions, Pleasanton, Cal.). In some embodiments, the adaptor ligated DNA may be separated from excess adaptors and unligated DNA.

The adaptor may further comprise features such as universal primer binding site (including a sequencing primer binding site) a barcode sequence (including a sample barcode (SID) or a unique molecular barcode or identifier (UID or UMI). In some embodiments, the adaptors comprise all of the above features while in other embodiments, some of the features are added after adaptor ligation by extending tailed primers that contain some of the elements described above.

The adaptor may further comprise a capture moiety. The capture moiety may be any moiety capable of specifically interacting with another capture molecule. Capture moieties -capture molecule pairs include avidin (streptavidin) – biotin, antigen – antibody, magnetic (paramagnetic) particle – magnet, or oligonucleotide – complementary oligonucleotide. The capture molecule can be bound to a solid support so that any nucleic acid on which the capture moiety is present is captured on solid support and separated from the rest of the sample or reaction mixture. In some embodiments, the capture molecule comprises a capture moiety for a secondary capture molecule. For example, a capture moiety in the adaptor may be a nucleic acid sequence complementary to a capture oligonucleotide. The capture oligonucleotide may be biotinylated so that adapted nucleic acid-capture oligonucleotide hybrid can be captured on a streptavidin bead.

In some embodiments, the adaptor-ligated nucleic acid is enriched via capturing the capture moiety and separating the adaptor-ligated target nucleic acids from unligated nucleic acids in the sample.

In some embodiments, the stem portion of the adaptor includes a modified nucleotide increasing the melting temperature of the capture oligonucleotide, e.g., 5-methyl cytosine, 2,6-diaminopurine, 5-hydroxybutynl-2′-deoxyuridine, 8-aza-7-deazaguanosine, a ribonucleotide, a 2′O-methyl ribonucleotide or a locked nucleic acid. In another aspect, the capture oligonucleotide is modified to inhibit digestion by a nuclease, e.g., by a phosphorothioate nucleotide.

In some embodiments, the invention utilizes a barcode. Detecting individual molecules typically requires molecular barcodes such as described in U.S. Pat. Nos. 7,393,665, 8,168,385, 8,481,292, 8,685,678, and 8,722,368. A unique molecular barcode is a short artificial sequence added to each molecule in the patient’s sample typically during the earliest steps of in vitro manipulations. The barcode marks the molecule and its progeny. The unique molecular barcode (UID) has multiple uses. Barcodes allow tracking each individual nucleic acid molecule in the sample to assess, e.g., the presence and amount of circulating tumor DNA (ctDNA) molecules in a patient’s blood in order to detect and monitor cancer without a biopsy (Newman, A., et al., (2014) An ultrasensitive method for quantitating circulating tumor DNA with broad patient coverage, Nature Medicine doi:10.1038/nm.3519).

A barcode can be a multiplex sample ID (MID) used to identify the source of the sample where samples are mixed (multiplexed). The barcode may also serve as a unique molecular ID (UID) used to identify each original molecule and its progeny. The barcode may also be a combination of a UID and an MID. In some embodiments, a single barcode is used as both UID and MID. In some embodiments, each barcode comprises a predefined sequence. In other embodiments, the barcode comprises a random sequence. In some embodiments of the invention, the barcodes are between about 4-20 bases long so that between 96 and 384 different adaptors, each with a different pair of identical barcodes are added to a human genomic sample. A person of ordinary skill would recognize that the number of barcodes depends on the complexity of the sample (i.e., expected number of unique target molecules) and would be able to create a suitable number of barcodes for each experiment.

Unique molecular barcodes can also be used for molecular counting and sequencing error correction. The entire progeny of a single target molecule is marked with the same barcode and forms a barcoded family. A variation in the sequence not shared by all members of the barcoded family is discarded as an artifact and not a true mutation. Barcodes can also be used for positional deduplication and target quantification, as the entire family represents a single molecule in the original sample (Newman, A., et al., (2016) Integrated digital error suppression for improved detection of circulating tumor DNA, Nature Biotechnology 34:547).

In some embodiments, the number of UIDs in the plurality of adaptors or barcode-containing primers may exceed the number of nucleic acids in the plurality of nucleic acids. In some embodiments, the number of nucleic acids in the plurality of nucleic acids exceeds the number of UIDs in the plurality of adaptors.

In some embodiments, the invention comprises intermediate purification steps. For example, any unused oligonucleotides such as excess primers and excess adaptors are removed, e.g., by a size selection method selected from gel electrophoresis, affinity chromatography and size exclusion chromatography. In some embodiments, size selection can be performed using Solid Phase Reversible Immobilization (SPRI) technology from Beckman Coulter (Brea, Cal.). In some embodiments, a capture moiety is used to capture and separate adaptor-ligated nucleic acids from unligated nucleic acids or excess primers from the products of exponential amplification.

The invention is a method of detecting genomic rearrangements in a sample using pairs of forward and reverse primers. The method comprises simultaneously interrogating the sample for more than one genomic rearrangement including more than one type of genomic rearrangement in a sample.

Referring to FIG. 1, the invention utilizes one or more pairs of a forward and a reverse oligonucleotide primers wherein orientation or proximity of the primers enables amplification of the intervening sequence if a rearrangement is present, but does not allow amplification if the rearrangement is not present.

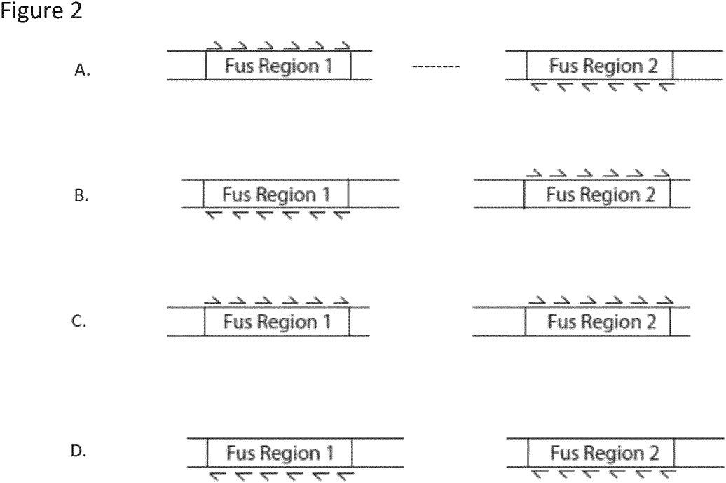

Referring to FIG. 2, the rearrangement is a gene fusion. In panel A, illustrating the reference genome sequence, the forward and reverse primers are annealing to opposite strands in a correct orientation but are not in proximity of each other (either too far on the same chromosome or are on different chromosomes. In a rearranged genome sequence, the forward and reverse primers anneal to sites that are in correct orientation and in proximity to each other and therefore enable amplification of the intervening sequence. In panel B, illustrating the reference genome sequence, the forward and reverse primers are annealing to the opposite strands but in an incorrect orientation and may or may not be in proximity of each other. In a rearranged genome sequence, the forward and reverse primers anneal to sites that are in correct orientation and in proximity to each other and therefore enable amplification of the intervening sequence. In panel C, illustrating the reference genome sequence, the forward and reverse primers are annealing to the same (+) strand and may or may not be in proximity of each other. In a rearranged genome sequence, the forward and reverse primers anneal to sites that are on opposite strands in correct orientation and in proximity to each other and therefore enable amplification of the intervening sequence. In panel D, illustrating the reference genome sequence, the forward and reverse primers are annealing the same (-) strand and may or may not be in proximity of each other. In a rearranged genome sequence, the forward and reverse primers anneal to sites that are on opposite strands in correct orientation and in proximity to each other and therefore enable amplification of the intervening sequence.

In some embodiments, (e.g., fusions of ALK, ROS or NTRK1 genes), the exact fusion partner is not known. In these instances, a primer or a series of tiled primers is designed to hybridize to multiple fusion candidates. Only the primers hybridizing to the fusion candidate actually involved in a gene fusion will enable amplifying the fusion breakpoint sequence. None of the primers annealing to other fusion candidates will yield an amplicon.

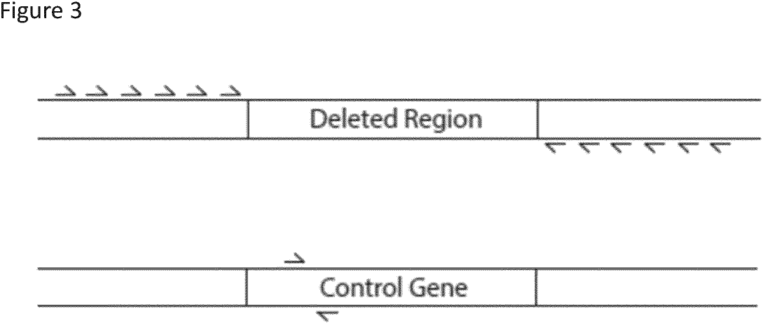

Referring to FIG. 3, the rearrangement is a deletion. In FIG. 3, illustrating the reference genome sequence, the forward and reverse primers are annealing to opposite strands in a correct orientation but are not in proximity of each other. In the rearranged genome sequence, the deletion brings the forward and reverse primer sites in proximity to each other to enable amplification of the intervening sequence. In this embodiment, a pair of control forward and reverse primers may be used. At least one in the pair of control forward and reverse primers anneals to a site in the reference genome, which is within the deleted region in the rearranged genome. Amplification of the intervening sequence is enabled in the reference genome but is not enabled in a rearranged genome. In some embodiments, the control forward and reverse primers anneal to a site of the genome unlikely to be involved in a copy number change such as a deletion or an amplification.

Notably, the method illustrated in FIG. 3 is suitable for detecting deletions of a variety of sizes. The size of the deleted region is taken into account and primers are placed so as to be too far apart in the reference genome to enable amplification of the intervening sequence.

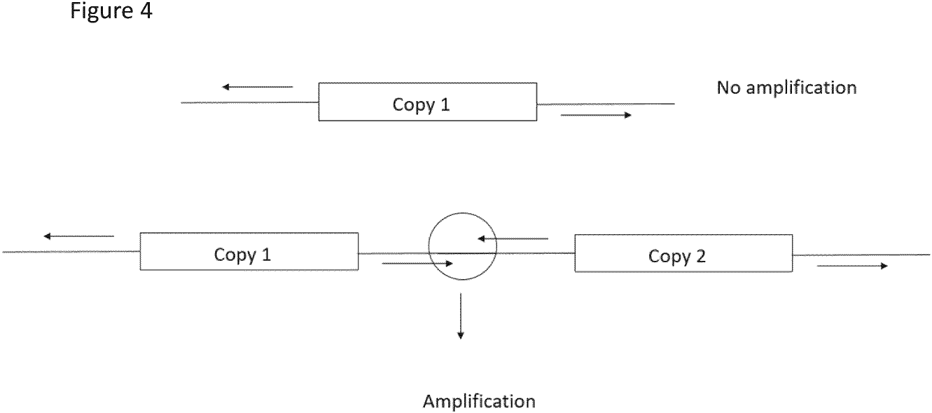

Referring to FIG. 4, the rearrangement is a duplication or a higher order gene amplification. In FIG. 4, top, illustrating the reference genome sequence, the forward and reverse primers are annealing to opposite strands but in an incorrect orientation. In the rearranged genome (FIG. 4, bottom), the tandem duplication (or higher level amplification) event brings at least one pair of the forward and reverse primer sites into correct orientation to enable amplification of the intervening sequence. Notably, the method illustrated in FIG. 4 is suitable for detecting duplication of a variety of sizes. The size of the expected duplication (or higher level amplification) is taken into account and primers are placed such that, in the absence of a rearrangement, they are in the wrong orientation and too far apart to enable amplification via PCR, but in the presence of a gene duplication (or higher level amplification), at least one pair of the forward and reverse primers is in the correct orientation and closely enough spaced to enable amplification.

The method further comprises, after exponential amplification with rearrangement-specific pairs of forward and reverse primers, forming a library of amplified nucleic acids and sequencing the nucleic acids in the library thereby detecting one or more genomic rearrangements in the sample.

In some embodiments, the method is multiplexed, meaning that the rearrangement-specific pairs of forward and reverse primers include multiple primer pairs positioned as illustrated on FIGS. 2, 3 and 4. The multiple primer pairs include one or more pairs detecting one or more gene fusions, one or more pairs detecting one or more gene deletions and one or more pairs detecting one or more gene amplifications. For example, the same reaction mixture may contain primer pairs targeting the fusions involving each of ALK, PPARG, BRAF, EGFR, FGFR1, FGFR2, FGFR3, MET, NRG1, NTRK1, NTRK2, NTRK3, RET, ROS1, AXL, PDGFRA, PDGFB , ABL1, ABL2, AKT1, AKT2, AKT3, ARHGAP26, BRD3, BRD4, CRLF2, CSF1R, EPOR, ERBB2, ERBB4, ERG, ESR1, ESRRA, ETV1, ETV4, ETV5, ETV6, EWSR1, FGR, IL2RB, INSR, JAK1, JAK2, JAK3, KIT, MAML2, MAST1, MAST2, MSMB, MUSK, MYB, MYC, NOTCH1, NOTCH2, NUMBL, NUT, PDGFRB, PIK3CA, PKN1, PRKCA, PRKCB, PTK2B, RAF1, RARA, RELA, RSPO2, RSPO3, SYK, TERT, TFE3, TFEB, THADA, TMPRSS2, TSLP, TY, BCL2, BCL6, BCR, CAMTA1, CBFB, CCNB3, CCND1, CIC, CRFL2, DUSP22, EPC1, FOXO1, FUS, GLI1, GLIS2, HMGA2, JAZF1, KMT2A, MALT1, MEAF6, MECOM, MKL1, MKL2, MTB, NCOA2, NUP214, NUP98, PAX5, PDGFB, PICALM, PLAG1, RBM15, RUNX1, RUNX1T1, SS18, STAT6, TAF15, TAL1, TCF12, TCF3, TFG, TYK2, USP6, YWHAE, AR, BRCA1, BRCA2, CDKN2A, ERB84, FLT3, KRAS, MDM4, MYBL1, NF1, NOTCH4, NUTM1, PRKACA, PRKACB, PTEN, RAD51B, and RB1.

In some embodiments, the forward and reverse primers are designed to accommodate short input nucleic acids. For example, cell-free DNA, including circulating tumor DNA (ctDNA) averages 175 bp in length. The forward and reverse primers or series of tiled forward primers and series of tiled reverse primers are placed to have no more than about 50 bases between in inner-most 3′-ends.

In some embodiments, the invention is a method of enriching for a sequence containing a genomic rearrangement by a Primer Extension Target Enrichment (PETE) method. Multiple versions of PETE have been described, see U.S. Application Ser. Nos. 14/910,237, 15/228,806, 15/648,146 and International Application Ser. No. PCT/EP2018/085727. Briefly, Primer Extension Target Enrichment (PETE) involves capturing nucleic acids with a first target-specific primer comprising a capture moiety and capturing the capture moiety thereby enriching the target nucleic acids. Any additional target-specific or adapter-specific primers hybridize to the enriched target nucleic acids. In other embodiments, PETE involves capturing nucleic acids by hybridizing and extending a first primer comprising a capture moiety and capturing the capture moiety thereby enriching the target nucleic acids, then, in hybridizing to the captured nucleic acids a second target-specific primer, extending the second target-specific primer thereby displacing the extension product of the first target-specific primer and retaining the further enriched target nucleic acid hybridized to the second primer extension product.

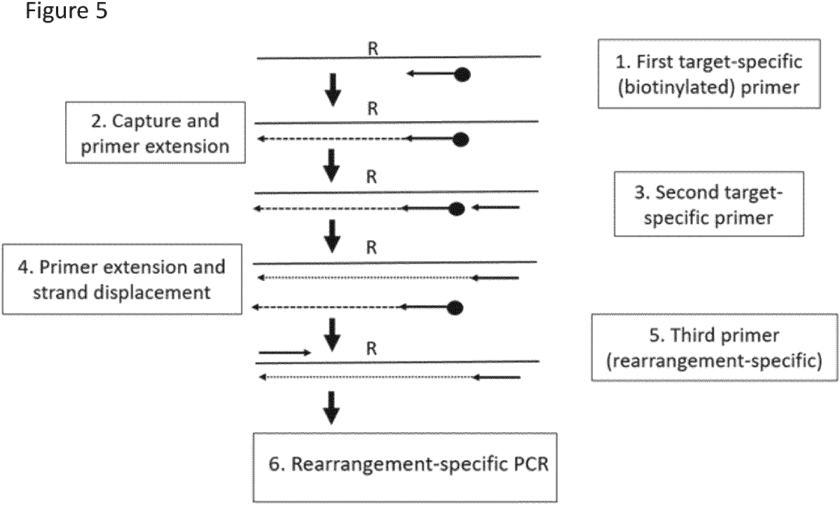

Referring to FIG. 5, one embodiment of the invention utilizes PETE. The method involves hybridizing to nucleic acids in a sample a first target-specific primer hybridizing on one side of a genomic rearrangement (R). (FIG. 5, step 1) The first primer comprises a capture moiety, e.g., biotin. Next, the first primer is extended and the hybridized first primer extension product (or earlier, the hybridized first primer) is captured via the capture moiety. The first primer extension product spans the site of the rearrangement (R) (FIG. 5, step 2).

The capture moiety on the first primer may be selected from a capture sequence, a chemical moiety for which a ligand is available (e.g., biotin) or an antigen for which an antibody is available. The capture sequence may be located in the 5′-portion of the first primer. It is a sequence complementary to a capture oligonucleotide. To improve capture, the capture oligonucleotide may comprise a modified nucleotide increasing the melting temperature of the hybrid between the capture oligonucleotide and the capture sequence in the first primer. The modified nucleotide is selected from 5-methyl cytosine, 2,6-diaminopurine, 5-hydroxybutynl-2′-deoxyuridine, 8-aza-7-deazaguanosine, a ribonucleotide, a 2′O-methyl ribonucleotide and locked nucleic acid.

The first primer bound to a solid support, e.g., a magnetic polymer-coated particle via the capture moiety prior to hybridizing the first oligonucleotide to the target nucleic acid so that the first primer extension complex is formed on solid support.

Next, the second target-specific primer hybridizes to the same strand of the target nucleic acid on the same side of the genomic rearrangement as to the first primer. (FIG. 5, step 3). The second primer may comprise a nucleic acid barcode or any other accessory sequence such as a universal primer binding site. The second primer is extended thereby producing a second primer extension complex and displacing the first primer extension product. The second primer extension product also spans the site of the rearrangement (R) (FIG. 5, step 4). Next, a third primer, hybridizes to the second primer extension product on the opposite side of the genomic rearrangement (FIG. 5, step 5). The third primer is designed in accordance with the instant disclosure to hybridize to a position suitable for exponential amplification in the rearranged genome but not in the reference genome. If the genomic rearrangement is present, the third primer and the second primer direct exponential amplification of the sequence containing the rearrangement site (FIG. 5, step 6). In some embodiments, an equivalent primer hybridizing to the second primer extension product o the same side of the rearrangement as the second primer is used instead of the second primer.

In some embodiments, the amplified rearrangement-specific nucleic acid is sequenced obtained by the target enrichment process is sequenced to determine or confirm the sequence of the rearrangement.

The nucleic acids and libraries of nucleic acids formed as described herein or amplicons thereof can be subjected to nucleic acid sequencing. Sequencing can be performed by any method known in the art. Especially advantageous is the high-throughput single molecule sequencing method utilizing nanopores. In some embodiments, the nucleic acids and libraries of nucleic acids formed as described herein are sequenced by a method involving threading through a biological nanopore (US10337060) or a solid-state nanopore (US10288599, US20180038001, US10364507). In other embodiments, sequencing involves threading tags through a nanopore. (US8461854) or any other presently existing or future DNA sequencing technology utilizing nanopores.

Other suitable technologies of high-throughput single molecule sequencing. include the Illumina HiSeq platform (Illumina, San Diego, Cal.), Ion Torrent platform (Life Technologies, Grand Island, NY), Pacific BioSciences platform utilizing the Single Molecule Real-Time (SMRT) technology (Pacific Biosciences, Menlo Park, Cal.) or a platform utilizing nanopore technology such as those manufactured by Oxford Nanopore Technologies (Oxford, UK) or Roche Sequencing Solutions (Santa Clara, Cal.) and any other presently existing or future DNA sequencing technology that does or does not involve sequencing by synthesis. The sequencing step may utilize platform-specific sequencing primers. Binding sites for these primers may be introduced in 5′-portions of the amplification primers used in the amplification step. If no primer sites are present in the library of barcoded molecules, an additional short amplification step introducing such binding sites may be performed. In some embodiments, the sequencing step involves sequence analysis. In some embodiments, the analysis includes a step of sequence aligning. In some embodiments, aligning is used to determine a consensus sequence from a plurality of sequences, e.g., a plurality having the same barcodes (UID). In some embodiments barcodes (UIDs) are used to determine a consensus from a plurality of sequences all having an identical barcode (UID). In other embodiments, barcodes (UIDs) are used to eliminate artifacts, i.e., variations existing in some but not all sequences having an identical barcode (UID). Such artifacts resulting from PCR errors or sequencing errors can be eliminated.

In some embodiments, the number of each sequence in the sample can be quantified by quantifying relative numbers of sequences with each barcode (UID) in the sample. Each UID represents a single molecule in the original sample and counting different UIDs associated with each sequence variant can determine the fraction of each sequence in the original sample. A person skilled in the art will be able to determine the number of sequence reads necessary to determine a consensus sequence. In some embodiments, the relevant number is reads per UID (“sequence depth”) necessary for an accurate quantitative result. In some embodiments, the desired depth is 5-50 reads per UID.

In some embodiments, the step of sequencing further includes a step of error correction by consensus determination. Sequencing by synthesis of the circular strand of the gapped circular template disclosed herein enables iterative or repeated sequencing. Multiple reads of the same nucleotide position enable sequencing error correction through establishment of a consensus call for each nucleotide or for the entire sequence or for a part of the sequence. The final sequence of a nucleic acid strand is obtained from the consensus base determinations at each position. In some embodiments, a consensus sequence of a nucleic acid is obtained from a consensus obtained by comparing the sequences of complementary strands or by comparing the consensus sequences of complementary strands. In some embodiments, the invention comprises after the sequencing step, a step of sequence read alignment and a step of generating a consensus sequence. In some embodiments, consensus is a simple majority consensus described in U.S. Pat. 8535882. In other embodiments, consensus is determined by Partial Order Alignment (POA) method described in Lee et al. (2002) “Multiple sequence alignment using partial order graphs,” Bioinformatics, 18(3):452-464 and Parker and Lee (2003) “Pairwise partial order alignment as a supergraph problem – aligning alignments revealed,” J. Bioinformatics Computational Biol., 11:1-18. Based on the number of iterative reads used to determine a consensus sequence, the sequence may be largely free or substantially free of errors.

In some embodiments, the rearrangement-specific amplicons and optional control amplicons formed according to the instant invention are detected without sequencing. The amplicons may be detected by end-point PCR, quantitative PCR (qPCR) or digital PCR (dPCR), including digital droplet PCR (ddPCR). In some embodiments, detection of genomic rearrangements is quantitative, such as the type of detection enabled by qPCR and dPCR. In other embodiments, detection of genomic rearrangements is qualitative, i.e., the read-out is the presence or absence of the rearrangement-specific amplification product on a gel electrophoresis or capillary electrophoresis.

In some embodiments, rearrangement-specific amplification according to the present invention is conducted by digital PCR (dPCR) including digital droplet PCR (ddPCR).

Digital PCR is a method of quantitative amplification of nucleic acids described e.g., in U.S. Pat. No. 9,347,095. The process involves partitioning a sample into reaction volumes so that each volume comprises one or fewer copies of the target nucleic acid. Each partition further comprises amplification primers, i.e., a forward and a reverse primer capable of supporting exponential amplification. In some embodiments, the partitioned reaction volume is an aqueous droplet.

In the context of the instant invention, the first primer of the forward and reverse primers is capable of hybridizing on one side of a genomic rearrangement, and a second primer of the forward and reverse primers is capable of hybridizing to the opposite strand on the opposite side of the genomic rearrangement relative to the first primer and adjacent to the first primer in the rearranged genome but not in a reference genome.

Each of the digital PCR reaction volumes further comprises a detectably-labeled probe capable of hybridizing to an amplicon of the first and second primers. The detectably labeled probe may be labeled with a combination of a fluorophore and the exponential amplification may eb performed with a nucleic acid polymerase having a 5′-3′-exonuclease activity.

In some embodiments, the method of the invention comprises performing an amplification reaction with the first and the second primers, wherein the reaction comprises a step of detecting the amplicon with the probe, and determining a number of reaction volumes where the probe has been detected thereby detecting the presence of a genomic rearrangement in the sample.

In some embodiments, the reaction volumes further comprise a third primer that is capable of hybridizing to the opposite strand relative to the first primer and adjacent to the first primer in the reference genome but not in the rearranged genome, and a second detectably labeled probe capable of hybridizing to the amplicon of the first and third primers but not the amplicon of the first and second primers. The second probe is distinct from the probe hybridizing to the amplicon of the first and second primers (the first probe). In such embodiments, the method further comprising determining a ratio of reaction volumes where the first probe has been detected to the number of reaction volumes where the second probe has been detected thereby detecting the frequency of genomic rearrangement.

1. A method of detecting a genomic rearrangement in a sample, the method comprising:
contacting a sample containing nucleic acids from a genome with one or more pairs of a forward and a reverse oligonucleotide primers, wherein the binding sites for the primers in a reference genome are not adjacent or not inward-facing, and wherein the position of the binding sites for the primers in a genome comprising a genomic rearrangement is adjacent and inward-facing to allow exponentially amplifying the nucleic acid comprising the rearrangement with the forward and reverse primers; and
exponentially amplifying the nucleic acid comprising the rearrangement thereby detecting the rearrangement.

2. The method of claim 1, further comprising sequencing the amplified nucleic acids thereby detecting the rearrangement.

3. The method of claim 1, wherein adjacent is less than 2000 base pairs apart in cellular genomic DNA.

4. The method of claim 1, wherein adjacent is less than 175 base pairs apart in cell-free DNA.

5. The method of claim 1, wherein the genomic rearrangement is a gene fusion and the binding sites for the forward and reverse primers are located on different chromosomes in a reference genome but are located on the same chromosome in the genome comprising the gene fusion.

6. The method of claim 1, wherein the genomic rearrangement is a deletion and the binding sites for the forward and reverse primers are located more than x base pairs apart in a reference genome but are located fewer than x bases apart in a genome comprising the deletion.

7. The method of claim 1, wherein the genomic rearrangement creates a breakpoint sequence and one of the binding sites for the forward and reverse primers spans the breakpoint sequence.

8. The method of claim 1, wherein the genomic rearrangement is an amplification and at least one of the copies of the forward primer binding site and one of the copies of the reverse primer binding site are inward-facing in the genome comprising the amplification.

9. A method of simultaneously interrogating a sample for one or more types of genomic rearrangements, the method comprising:
(a) contacting a sample containing nucleic acids from a genome with one or more pairs of a forward and a reverse oligonucleotide primers, wherein the binding sites for the primers in a reference genome are not adjacent or not inward-facing, and wherein the position of the binding sites for the primers in a genome comprising a genomic rearrangement is adjacent and inward-facing to allow exponentially amplifying the nucleic acid comprising the rearrangement with the forward and reverse primers;
(b) exponentially amplifying the nucleic acid comprising the rearrangement;
(c) forming a library of amplified nucleic acids; and
(d) sequencing the nucleic acids in the library thereby detecting one or more genomic rearrangements in the sample.

10. The method of claim 9, further comprising aligning the sequencing reads from step (d) with the reference genome to determine the genomic source of the genomic rearrangement.

11. The method of claim 9, wherein one or more pairs of a forward and a reverse oligonucleotide primers comprise:
(a) for at least one pair of forward and reverse primers, the binding sites for the forward and reverse primers are located on different chromosomes in a reference genome but are located on the same chromosome in the genome comprising a gene fusion; and
(b) for at least one pair of forward and reverse primers, one of the binding sites for the forward and reverse primers spans a breakpoint sequence of a genomic rearrangement; and
(c) for at least one pair of forward and reverse primers, one of the copies of the forward primer binding site and one of the copies of the reverse primer binding site are inward-facing in the genome comprising gene amplification.

12. A method of detecting a genomic rearrangement in a sample, the method comprising:
(a) forming a library of nucleic acids comprising at least one adaptor;
(b) hybridizing to a library nucleic acid a first primer of a primer pair, wherein the first primer hybridizes on one side of a genomic rearrangement and also comprises a capture moiety;
(c) extending the hybridized first primer, thereby producing a first primer extension complex comprising the sequence of the genomic rearrangement and further comprising a capture moiety
(d) capturing the first primer extension product via the capture moiety;
(e) hybridizing to the captured nucleic acid a second primer of a primer pair wherein second primer hybridizes to the opposite strand on the opposite side of the genomic rearrangement relative to the first primer and adjacent to the first primer in the rearranged genome but not in the reference genome;
(f) forming a copy of the captured rearranged nucleic acid; and
(g) sequencing the copy of the rearranged nucleic acid thereby detecting the genomic rearrangement.

13. A method of enriching for a sequence containing a genomic rearrangement in a sample, the method comprising:
(a) hybridizing to nucleic acids in a sample a first primer, wherein the first primer hybridizes on one side of a genomic rearrangement and also comprises a capture moiety;
(b) extending the hybridized first primer, thereby producing a first primer extension complex comprising the sequence of the genomic rearrangement and further comprising the capture moiety;
(c) capturing the first primer extension product via the capture moiety;
(d) hybridizing to the captured nucleic acid a second primer, wherein second primer hybridizes to the same strand on the same side of the genomic rearrangement relative to the first primer in the rearranged genome but not in the reference genome, and also comprises a barcode;
(e) extending the hybridized second primer, thereby producing a second primer extension complex and displacing the first primer extension complex comprising the capture moiety;
(f) hybridizing to the second primer extension complex a third primer wherein the third primer hybridizes to the opposite strand on the opposite side of the genomic rearrangement relative to the second primer and adjacent to the second primer in the rearranged genome but not in the reference genome; and
(g) extending the third primer thereby forming a double-stranded product comprising the sequence of a rearrangement thereby enriching for the genomic rearrangement.

14. A method of detecting a structural variation in RNA transcripts in a sample, comprising:
(a) obtaining nucleic acids from a sample;
(b) reverse transcribing RNA transcripts into cDNA strands with a first primer positioned adjacent to a site of a genomic rearrangement;
(c) hybridizing to the cDNA strands a second primer wherein the second primer hybridizes to the opposite strand on the opposite side of the genomic rearrangement relative to the first primer and adjacent to the first primer in a rearranged genome but not in a reference genome to enable exponential amplification of a rearranged genome sequence but not of a reference genome sequence; and
(d) amplifying the cDNA to produce amplicons thereby detecting genomic rearrangement in the RNA transcripts.

15. A method for detecting a genomic rearrangement in a nucleic acid in a sample, comprising:
(a) partitioning a sample comprising nucleic acids from a genome into a plurality of reaction volumes; wherein each reaction volume comprises (i) a first primer that is capable of hybridizing on one side of a genomic rearrangement, (ii) a second primer that is capable of hybridizing to the opposite strand on the opposite side of the genomic rearrangement relative to the first primer and adjacent to the first primer in the rearranged genome but not in a reference genome, and (iii) a detectably-labeled first probe capable of hybridizing to an amplicon of the first and second primers;
(b) performing an amplification reaction with the first and the second primers, wherein the reaction comprises a step of detection with the probe; and
(c) determining a number of reaction volumes where the first probe has been detected thereby detecting the genomic rearrangement.
